(19) United States
(12) Patent Application Publication
(10) Pub. No.: US 2024/0164906 A1
(43) Pub. Date: May 23, 2024

(54) NEW GUIDED BONE REGENERATION DEVICE

(71) Applicant: BIOTECH DENTAL, Salon-de-Provence (FR)

(72) Inventor: Pierre KELLER, Strasbourg (FR)

(21) Appl. No.: 18/516,353

(22) Filed: Nov. 21, 2023

(30) Foreign Application Priority Data
Nov. 21, 2022 (FR) 2212116

(51) Int. Cl. A61F 2/28

(52) U.S. Cl. CPC A61F 2/2803; A61F 2002/30985; A61F 2310/00023

(57) ABSTRACT

A device for guided bone regeneration includes a custom-made covering plate that covers a bone defect, whose shape allows for the surface of the bone to be restored as it was prior to the appearance of the bone defect. The covering plate rests on the healthy bone surrounding the bone defect and is made of grade 1 or grade 2 titanium.

This patent application claims the priority of French patent application FR2212116 filed on Nov. 21, 2022.

### FIELD OF THE INVENTION

The present invention relates to the field of devices intended for guided bone regeneration.

### PRIOR ART

The destruction of bone tissue can sometimes occur due to infection, cancer, or trauma. Such destruction is normally followed by bone healing. In certain cases, this bone healing may prove insufficient to completely regenerate the destroyed tissue, resulting in the appearance of bone defects. Such bone defects correspond to spaces or cavities in place of the destroyed bone.

Faced with such defects, it is possible to use biomaterials to fill a bone defect and promote bone healing within it.

Typically, the biomaterial used consists of either synthetic bone substitute material (e.g., hydroxyapatite granules) or bone particles. Once the space corresponding to the bone defect is filled with this biomaterial, this space is then closed and covered by a covering device directing the biomaterial growth.

Two types of covering devices have been developed in the prior art.

A first device type corresponds to the covering membranes used since the beginning of the 1960s in orthopedic bone surgery. These woven or non-woven membranes can have multiple structures, whether in the form of simple membranes (e.g. U.S. Pat. No. 4,961,707), or membranes based on synthetic multilayers (e.g.: U.S. Pat. No. 4,816,339), titanium mesh (e.g. utility model RU 152119 U1), or even with hybrid compositions (synthetic and titanium).

These covering membranes have interesting characteristics, since:

1—They are easy to install by the practitioner and stabilize the bone graft and the clot.

2—They oppose the resorption of the graft which, without a membrane, can lose up to 25% of its volume after 4 months.

3—They prevent the proliferation of cells from the covering mucosa and therefore promote the migration of cells from the marrow spaces into the clot inhabiting the site to be regenerated.

4—They can finally be removed without difficulty (now some are resorbable).

Now, these covering membranes have the disadvantage of having a very great flexibility. If this flexibility facilitates their installation, it does not allow them to maintain the regeneration volume as delimited by the practitioner at the time of their installation. In fact, these covering membranes are subjected to stresses such as those resulting from the patient chewing on their corresponding area.

A second type of covering devices developed from the 1990s consisted of titanium covering plates (rigid and perforated) as described in the patent EP 2536446 B1, the patent EP 3294222 B1 or in the patent RU 2 748 200 C1.

These covering plates also have interesting characteristics, since:

1—They are accessible for installation due to their large meshes allowing them to be shaped by the practitioner. Now, they require certain know-how.

2—They partially oppose the resorption of the graft.

3—Their rigidity makes it possible to stably over time delimit the bone regeneration volume, even during episodes of patient chewing.

In addition to their more delicate installation than the membranes and the resorption of graft which can be associated with them, these covering plates have the disadvantage of not blocking the proliferation of cells from the covering mucosa and especially of present a very open structure (mesh) which can make their removal difficult after osteo-regeneration due to the risk of tearing of tissues which could have developed and/or anchored within their meshes.

These two devices therefore have mutual and, a priori, opposing advantages and disadvantages.

In the past, some have tried to couple these two devices to benefit from their respective advantages (e.g. U.S. Pat. No. 4,961,707), but this combination results in a greater complexity.

### SUMMARY OF THE INVENTION

Faced with this problem, the inventors have now developed a covering plate with sufficient rigidity to give a shape correcting the bone defect and to resist the patients chewing episodes, while simultaneously presenting an elasticity allowing it to be simply adjusted to the bone defect during its installation by the practitioner.

To do this, the inventors have developed a covering device made specifically from grade 1 or 2 titanium.

Titanium is a lustrous transition metal with low density and high strength. “Pure” titanium is therefore stronger than ordinary low-carbon steels, while being 45% lighter. The integration of titanium into different alloys allows them to be associated with particularly interesting properties. Thus, titanium alloys are generally used for most applications with small amounts of aluminum and vanadium, typically 6% and 4% respectively, by weight.

Solide titanium and its alloys can be found in two crystalline forms, namely compact hexagonal (a phase and below 880 0C for “pure” titanium) and/or centered cubic (β phase; above 880 0C for “pure” titanium). This dimorphism is the basis of a classification corresponding to the “alpha”, “alpha-beta” and “beta” families, each with specific usage properties. Thus, the highest mechanical strengths are obtained with ß alloys while “pure” titanium belongs to the alpha family. Now, it is possible to carry out a heat treatment called “annealing” on pure titanium or one of its alloys. This treatment corresponds to a temperature-controlled rise of titanium or one of its alloys so as to cause a transition from a crystalline structure in phase a to a crystalline structure in phase B. Now, the stability of this transition can vary with time.

Now, another classification exists for titanium. This classification uses “grades”, which relates to the purity of the titanium. In detail, we will talk about “pure” titanium for grade 1 to 4 titanium and grade 5 titanium and beyond for titanium alloys. In fact, titanium is never strictly pure, and we always find traces of other elements (oxygen and other components) within it. Thus, the purest titanium is grade 1 titanium, but it still contains 0.18% oxygen. Grade 4 titanium is the least pure titanium (of the 4 grades) and contains 0.4% oxygen.

Now, the properties of these different grades of titanium also differ beyond their composition in their properties, particularly with regard to ductility and mechanical strength.

Ultimately, grade 1 titanium is simply not used in implantology because too ductile. Grade 2 titanium has certainly been used in the past. Now, grade 4 titanium is preferred in implantology and serves as the basis for current covering plates, because of better mechanical properties. Note that grade 5 titanium (alloy with niobium), whose hardness is greater than grade 4 titanium, is today used for certain prosthetic components. For reviews on the current use of titanium and its alloys in implantology, one can consult LIU et al. (Regenerative biomaterials, vol. 4, page 315 to 323, 2017) or NICHOLSON (Prosthesis, vol. 2, page 100 to 116, 2020).

On the basis of the two types of known covering devices, the inventor did not choose a simple combination. Conversely, he changed the structure of the covering plate—i.e. to be microperforated instead of perforated—, and the used material—i.e. to grade 1 or 2 titanium instead of grade 4 titanium—. The obtained covering plate then presents the advantages of both types of known covering devices, while being simple in design.

The first object of the invention relates to a device (1) intended for guided bone regeneration which comprises at least one covering plate whose shape allows for the recreation of a bone's surface as it was prior to the appearance of a bone defect by covering said bone defect while resting, at least partially, on the healthy bone surrounding said bone defect, characterized in that said covering plate is manufactured using grade 1 or 2 titanium.

In order to facilitate installation by the practitioner and provide maximum comfort to the patient, the covering plate is tailored to the patient's anatomy. Typically, this covering plate will be custom made, for example, on the basis of a mold of the bone defect to be corrected.

A second object of the invention relates to a covering plate as defined previously.

Advantageously, the covering plate of the invention has a thickness from 0.1 to 1.5 mm, and preferably from 0.3 to 1 mm.

Again advantageously, the surface of the covering plate has microperforations. which have a diameter of between 10 and 500 μm, preferably between 50 and 100 μm.

### DESCRIPTION OF THE DRAWINGS

FIG. 1 is a representation of one embodiment of a covering plate of a device according to the invention.

### DETAILED DESCRIPTION OF THE INVENTION

The covering plate may have a rounded or rectangular shape or, more generally, any shape adapted to the targeted bone surface. Therefore, and especially in the case of a rectangular covering plate, care will be taken to round the corners and edges so as not to risk injuring the periosteum or soft tissues.

The dimensions of this covering plate will be such that it completely covers the bone defect further with a minimum of 50% of its surface resting on the healthy bone framing said bone defect, preferably at least 10% of its surface.

Typically, a bone defect presents the following:
a length between 5 and 200 mm, preferably between 10 and 100 mm.
a width of between 3 and 20 mm, preferably between 5 and 10 mm.
a depth of between 5 and 20 mm, preferably between 8 and 15 mm.

According to a preferred embodiment, the bone defect is a bone defect of the jaw.

To obtain these particularly advantageous properties, the covering plate of the device according to the invention has a thickness from 0.1 to 1.5 mm, and preferably from 0.3 to 1 mm.

Even if the covering plate can be obtained by any method known to those of ordinary skill in the art, such as molding or milling, the use of 3D printing for its manufacture is preferable. Such 3D printing typically uses a metal powder whose shaping into a covering plate is carried out by a laser of suitable power.

According to a preferred embodiment, the surface of the covering plate has microperforations. These microperforations allow for the supplying of nutrients to the regenerating bone in the bone defect by allowing these nutrients to pass through the entire thickness of the plate. Furthermore, their size also simultaneously prevents colonization of the covering plate by tissues (soft and/or hard), thereby facilitating the removal of the covering plate if necessary.

By microperforations we mean perforations having a diameter of between 10 and 500 μm, preferably between 50 and 100 μm.

It is preferable for the portion of the covering plate that rests on the healthy bone surrounding the bone defect to be made of solid material (and therefore without microperforations on its surface).

Typically, this surface made of solid material constitutes a strip that forms all or part of the edge of the covering plate. The width of this edge is between 0.5 and 5 mm, preferably between 1 and 2 mm.

According to another preferred embodiment, the device according to the invention further comprises at least one fixation means.

Typically, said fixation means can take the form of a pin, a screw, a nail, or a glue adapted to the bone.

Advantageously, said fixation means takes the form of a screw and the covering plate then preferably comprises a drilling adapted to the head of said screw. Preferably, the drilling is positioned on the cover plate so as to coincide with an area of healthy bone surrounding the bone defect.

Again advantageously and in the case where the covering plate is intended for guided bone regeneration of a bone defect of the jaw, said covering plate do not include any screw holes positioned on the side of either the palatal crest or the lingual cavity.

Indeed, the characteristics of the grade 1 or grade 2 titanium covering plate allow it to be positioned correctly without requiring multiple attachment points. It is thus possible to place without being attached to the jawbone on the side of either the palatal crest or the lingual cavity, which are difficult to access for the practitioner.

The examples which follow are provided for illustration purposes and cannot limit the scope of the present invention.

Bone Regeneration of a Bone Defect in the Jaw with a View to Placing Dental Implants

In the context of a bone defect in the jaw, it is necessary to first allow for the regeneration of this bone defect in order to be able to determine the proper placement of dental implants.

To do this, an incision is made in the gum in order to access this bone defect. This opening is made by raising the flap of gum resulting from the incision. The practitioner can then apply the material intended for bone regeneration to the bone defect, in particular by using the Sausage Technique™ of Professor Istvan Urban.

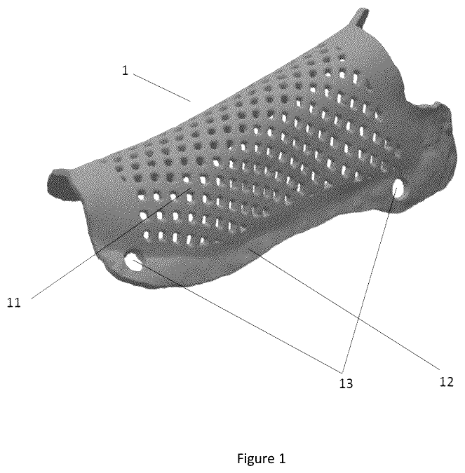

FIG. 1 shows a covering plate (1) made of grade 2 titanium that has been 3D printed. This covering plate (1) has a central surface with microperforations positioned above the bone defect (11). This covering plate (1) also has a peripheral surface taking the form of a strip of solid, unperforated material (12) that will rest on the healthy bone surrounding the bone defect.

This covering plate (1) also has two holes (13), located in the solid material of the peripheral surface (12) of the covering plate. Neither of these two holes (13) are positioned on the side of either the palatal crest or the lingual cavity in relation to the bone defect that requires regeneration.

This covering plate (1) can thus be placed and attached to the healthy bone of the jaw surrounding the bone defect by the practitioner by means of screws positioned in these two holes (13) and fixed to the healthy bone surrounding the bone defect.

Closure of the flap over the covering plate can then be carried out by the practitioner via suturing (preferably absorbable).

After healing, bone regeneration can occur under the covering plate for a period of up to several months or even a year.

After this regeneration period, the covering plate (1) can be removed without compromising the structure, and thus the solidity, of the regenerated bone. Indeed, its microperforations prevent soft and hard tissues from adhering to it.

In this way, the removal of the covering plate (1) according to the invention compromises neither the structure nor the solidarity of the regenerated bone. It is then possible to place dental implants in this regenerated bone.

1. A device intended for guided bone regeneration which comprises at least one covering plate whose shape permits the recreation of a bone's surface as the bone's surface was prior to the appearance of a bone defect by covering said bone defect while resting, at least partially, on the healthy bone surrounding said bone defect, wherein said covering plate is made of grade 1 or grade 2 titanium.

2. The device of claim 1, wherein the bone defect is a bone defect of the jaw.

3. The device of claim 1, wherein the covering plate of the device according to the invention has a thickness between 0.3 and 1 mm.

4. The device of claim 1, wherein the covering plate is manufactured by 3D printing.

5. The device of claim 1, wherein the surface of the covering plate has micro-perforations that correspond to holes having a diameter between 10 and 500 μm.

6. The device of claim 1, wherein the surface of the covering plate has micro-perforations that correspond to holes having a diameter between 50 and 100 μm.

7. The device of claim 1, wherein the covering plate is made of solid, unperforated material on the part of the covering plate's surface that rests on the healthy bone surrounding the bone defect.

8. The device of claim 7, wherein the surface made of solid material constitutes a strip which forms all or part of the peripheral edge of the covering plate and whose width is between 0.5 and 5 mm.

9. The device of claim 7, wherein the surface made of solid material constitutes a strip which forms all or part of the peripheral edge of the covering plate and whose width is between 1 and 2 mm.

10. The device of claim 1, wherein said device further comprises at least one fixation means.

11. The device of claim 10, wherein said at least one fixation means takes the form of a screw and the covering plate then comprises at least one drilling adapted to the head of said screw.

12. A covering plate whose shape permits the recreation of a bone's surface as the bone's surface was prior to the appearance of a bone defect by covering said bone defect while resting, at least partially, on the healthy bone surrounding said bone defect, wherein said covering plate is made of grade 1 or grade 2 titanium.

13. The covering plate of claim 12, wherein said covering plate is manufactured by 3D printing.

14. The covering plate of claim 12, wherein the covering plate's surface has micro-perforations that correspond to holes having a diameter between 10 and 500 μm.

15. The covering plate of claim 12, wherein the covering plate's surface has micro-perforations that correspond to holes having a diameter between 50 and 100 μm.

16. The covering plate of claim 12, wherein said covering plate is made of solid, unperforated material on the part of the covering plate's surface that rests on the healthy bone surrounding the bone defect.

17. The covering plate of claim 16, wherein the covering plate's surface made of solid material constitutes a strip which forms all or part of the peripheral edge of the covering plate and whose width is between 0.5 and 5 mm.

18. The covering plate of claim 16, wherein the covering plate's surface made of solid material constitutes a strip which forms all or part of the peripheral edge of the covering plate and whose width is between 1 and 2 mm.

19. The covering plate of claim 12, wherein said covering plate comprises at least one drilling.
